(19) United States
(12) Patent Application Publication
(10) Pub. No.: US 2012/0108868 A1
(43) Pub. Date: May 3, 2012

(54) Process for the Production of Paraxylene

(76) Inventors: Dana Lynn Pilliod, League City, TX (US); John Di-Yi Ou, Houston, TX (US)

(21) Appl. No.: 13/279,665

(22) Filed: Oct. 24, 2011

Related U.S. Application Data
(60) Provisional application No. 61/408,097, filed on Oct. 29, 2010.

(51) Int. Cl. C07C 5/27; B01J 14/00

(52) U.S. Cl. 585/300; 422/187

(57) ABSTRACT

A process for the purification of aromatic feedstream to produce paraxylene is disclosed, including the separation of a C8+ aromatic feedstream into a steam comprising C8 aromatic species and a stream comprising C9+ aromatic species. After separation of PX from the C8 aromatic stream, a PX-depleted stream is separated and processed in a liquid phase isomerization unit and a vapor phase isomerization unit in parallel.

### PRIORITY CLAIM

This application claims the benefit of Provisional Application No. 61/408,097, filed Oct. 29, 2010.

### FIELD OF THE INVENTION

The invention relates to a process for producing paraxylene including xylene isomerization, and to an apparatus for the practice of said process.

### BACKGROUND OF THE INVENTION

The xylene isomers are important intermediates, which find wide and varied application in chemical syntheses. By way of example, paraxylene (PX) is a feedstock for terephthalic acid which finds use in the manufacture of synthetic fibers; metaxylene (MX) is used in the manufacture of dyes, and orthoxylene (OX) is used as a feedstock for phthalic is anhydride, which finds use in the manufacture of plasticizers.

Xylenes are found in various fractions such as coal tar distillate, petroleum reformates and pyrolysis liquids in admixture with other compounds of like boiling point. The aromatic components are readily separated from non-aromatics by methods such as solvent extraction. A fraction may then be obtained readily such as by distillation, consisting essentially of C8 aromatics. By “C8 aromatics” is meant aromatic hydrocarbons having 8 carbon atoms, including particularly ethylbenzene and the xylene isomers paraxylene (p-xylene or PX), orthoxylene (o-xylene or OX), and metaxylene (m-xylene or MX).

While difficult to separate, due to their similar chemical structures, physical properties, and identical molecular weights, there are various methods used to separate the C8 isomers, for instance OX is separable from other C8 aromatics by fractional distillation, and PX is separable by fractional crystallization or selective sorption. Present demand is largely for PX and it is desirable to convert MX, the principal xylene present in the feed stream, and also OX to PX, to meet the market demand. At ordinary temperatures at which xylenes are processed in a typical petrochemical plant, the thermodynamic equilibrium content is approximately 24 mol % PX, 56 mol % MX, and 20 mol % OX, based on the total amount of xylenes in said feed.

The production of paraxylene in a conventional aromatics complex is energy intensive. This is due in part to the significant amount of recycle that is reprocessed through the C9+ removal. A typical commercial process is illustrated in FIG. 1, which shows processing of a stream comprising C8+ aromatics (C8 aromatic hydrocarbons and higher carbon number aromatic hydrocarbons) with the object being, at least in part, recovery of a stream comprising PX in an amount greater than that found in an equilibrium mixture of xylenes.

The feed stream or streams used in the system shown in FIG. 1 may come from a variety of sources, such as one or more sources selected from C8+ Reformate 1, C8+ Selective Toluene Disproportionation Product 17, C8+ Transalkylation Product 2, C8+ Toluene Disproportionation Product 15, and any other streams that contain C8 aromatics, such as products from toluene methylation with methanol. Each of these sources is per se well-known in the art. These streams typically comprise the four C8 isomers and heavier aromatics (C9+ aromatics) which are processed along with a recycle stream 10, in one or more fractionators in C8/C9+ Aromatics Fractionation 16 to remove C9+ aromatics into Stream 3. The C9+ and heavier aromatics could have an adverse effect on downstream is Paraxylene Recovery 12 and Xylenes Isomerization 13 if not removed from the feed stream(s) by fractionation.

The C8/C9+ Aromatics Fractionation in 16 thus yields C8 Aromatics stream 6 which typically contains between 10 and 95 wt % paraxylene, and bottoms product 3 comprising C9+ aromatics. The C8 aromatics stream 6 is processed to selectively recover paraxylene by one or both of selective adsorption or crystallization which is shown as Paraxylene Recovery unit 12 in FIG. 1. A Paraxylene Product, which may comprise as much as 99.7 wt % or even higher of paraxylene is recovered as Stream 7, with the balance of C8 aromatics passing via conduit 8 to Vapor Phase Xylenes Isomerization 13. Optionally, in the presence of hydrogen provided by Stream 9, Vapor Phase Xylenes Isomerization 13 establishes a near-equilibrium balance of xylene isomers in Stream 19 using one or more of a variety of catalysts which may also convert ethylbenzene to benzene and ethane or may convert ethylbenzene to near-equilibrium xylene isomers. Vapor phase processes and catalysts therefore are per se well-known in the art.

Continuing with the system shown in FIG. 1, the Xylenes Isomerization Product 19 passes to Detoluenization Fractionation 18 which removes C7 and lighter materials (C7−) in Stream 11 to yield Isomerate Recycle Stream 10. Isomerate Recycle Stream 10 is recycled to the C8/C9+ Aromatics Fractionation 16.

Improving such energy-intensive processes is an active area of research, but it is not a simple matter of optimization of each individual step, as optimization of one step may negatively affect one or more steps in the overall system. Examples of proposed improvements include the following.

U.S. Pat. No. 3,856,874 describes splitting the effluent stream from PX separation, passing the independent streams over different catalysts, then combining the isomerized streams and recycling.

U.S. Pat. No. 7,439,412 teaches a process for recovering one or more high purity xylene isomers from a C8+ aromatic feedstream, including the use of an isomerization unit under liquid phase conditions. In an example, the product of the liquid phase isomerization unit is returned to the first fractionation tower in the system. See also U.S. Pat. No. 7,626,065.

U.S. Pat. No. 7,553,998 teaches a process for recovering one or more high-purity xylene isomers from a feed having substantial content of C9+ aromatic hydrocarbons is comprising de-ethylation of heavy aromatics followed by fractionation and then passing the stream to a C8 aromatic isomer recovery to recover high-purity xylene isomer with lowered energy costs. Streams passing through an isomerization unit under liquid isomerization conditions are split, with a portion sent to an isomer recovery unit and a portion is purged.

U.S. application Ser. No. 12/612,007 (published as 2010/0152508) describes a process for producing a PX-rich product, the process comprising: (a) providing a PX-depleted stream; (b) isomerizing at least a portion of the PX-depleted stream to produce an isomerized stream having a PX concentration greater than the PX-depleted stream and a benzene concentration of less than 1,000 ppm and a C₉+ hydrocarbons concentration of less than 5,000 ppm; and (c) separating the isomerized stream by selective adsorption.

Provisional Application No. 61/326,445, filed Apr. 21, 2010, is directed to a xylenes isomerization process, including a liquid phase isomerization, for the production of equilibrium or near-equilibrium xylenes, wherein the process conditions include a temperature of less than 295° C. and a pressure sufficient to maintain the xylenes in liquid phase.

Other references of interest include U.S. Publication Nos. 2008/0262282; 2009/0149686; 2009/0182182; U.S. Pat. Nos. 6,448,459; 6,872,866; and 7,368,620.

The present inventors have surprisingly discovered a process which significantly reduces the energy required to produce high purity xylene isomers by providing parallel configuration of vapor phase and liquid phase isomerization systems.

### SUMMARY OF THE INVENTION

The invention is directed to a process for producing paraxylene comprising first separating a feed comprising C8+ aromatics into an overhead or first stream comprising xylene isomers and a bottoms product or second stream comprising C9+ aromatics, separating the xylene stream in a PX recovery unit to recover a PX-rich stream and a PX-depeleted stream, then separating said PX depleted (C8 aromatics) stream through a parallel configuration of vapor phase xylenes isomerization and liquid phase xylenes isomerization.

In embodiments, a benzene separation step occurs between the C8/C9+ fractionation and the PX recovery unit, and/or a benzene separation step downstream from the isomerization step(s). There may also be, in embodiments, a toluene separation step, such as downstream of said isomerization step(s).

In embodiments, the liquid phase isomerization product is recycled to one or more of the C8/C9+ fractionation, the benzene separation step (where present) and the PX recovery step.

The invention also relates to an apparatus for the production of paraxylene comprising a first fractionation column operating at conditions suitable for the separation of a C8+ aromatics stream into an overheads comprising xylenes and a bottoms product comprising C9+ aromatics, the overheads stream fluidly connected with a PX recovery unit, wherein said PX recovery unit provides a PX-enriched stream and a PX-depleted stream, the improvement comprising dividing a conduit carrying said PX-depleted stream so that a portion of said PX-depleted stream is passed to a vapor phase isomerization unit and another portion of said PX-depleted stream is passed to a liquid phase isomerization unit.

In embodiments, said liquid phase isomerization unit is fluidly connected so as to provide liquid phase isomerate recycle to said first fractionation column and/or to said PX recovery unit.

In embodiments, said PX recovery unit is selected from at least one of a crystallizer and an adsorptive separator.

In embodiments, at least one other fractionator upstream of said first fractionator, wherein said at least one other fractionator operates under conditions suitable for removing benzene from a stream comprising xylenes or for removing toluene from a stream comprising xylenes, and optionally wherein both said fractionator for removing benzene and said fractionator for removing toluene are provided upstream of said first fractionator.

It is an object of the invention to significantly reduce the energy required to produce paraxylene by minimizing the amount of isomerate recycle from vapor phase xylenes isomerization.

These and other objects, features, and advantages will become apparent as reference is made to the following detailed description, preferred embodiments, examples, and appended claims.

### BRIEF DESCRIPTION OF THE DRAWINGS

In the accompanying drawings, like reference numerals are used to denote like parts throughout the several views.

FIG. 1 is a schematic illustrating a prior art flow configuration for xylenes isomerization.

FIG. 2 is a schematic illustrating an embodiment of the invention.

FIGS. 3 and 4 represent a comparison of two systems, each embodiments of the present invention, the former returning liquid isomerization product to the rerun tower and is the latter returning liquid isomerization product to PX recovery unit.

### DETAILED DESCRIPTION

According to the invention, a hydrocarbon stream comprising C8+ aromatics is separated into a stream comprising C8 aromatics and a stream comprising C9+ aromatics. The C8 stream, optionally passed through a benzene separation unit, is then passed to a PX recovery unit to provide two streams, one having an increased PX concentration and the other having a decreased PX concentration. The PX-depleted stream is then divided and then isomerized, in parallel, in at least one liquid isomerization unit and in at least one vapor phase isomerization unit.

The invention may be better understood by reference to the specific embodiment illustrated in FIG. 2. It will be understood by one of skill in the art in possession of the present disclosure that numerous modifications can be made and that the embodiment should not be taken as limiting to the invention described in the appended claims.

The feed stream(s) to the system shown in FIG. 2 may come from one or more sources comprising C8+ aromatic hydrocarbons, including C8+ reformate 1, C8+ Selective Toluene Disproportionation Product 17, C8+ transalkylation product 2, C8+ toluene disproportionation product 15, and any other streams that contain C8 aromatics such as products from toluene methylation with methanol.

In the flow configuration shown in FIG. 2, energy consumption is reduced by minimizing the amount of Isomerate Recycle 10 from Vapor Phase Xylenes Isomerization 13 and controlling the amount of C9+ aromatics that are processed in the C8/C9+ Aromatics Fractionation 16. The new process configuration includes the parallel processing of Paraxylene Depleted C8 Aromatics Stream 8 through Vapor Phase Xylenes Isomerization and Liquid Phase Xylenes Isomerization 20. The flow of Paraxylene Depleted C8 Aromatics Stream 8 is minimized through Vapor Phase Xylenes Isomerization 13 to minimize energy by reducing the amount of Paraxylene Depleted C8 Aromatics Stream 30 that is vaporized in Vapor Phase Xylenes Isomerization 13 and the associated amount of Isomerate Recycle Stream 10, which contains a much higher concentration of by-product C9+ aromatics than Liquid Phase Xylenes Isomerization product. LP Isomerate Recycle Stream 21 which is the product from Liquid Phase Xylenes Isomerization 20 is sent to C8/C9+ Aromatics Fractionation 16 at a higher feed location to minimize energy consumption due to its lower concentration of C9+ aromatics. The amount of energy savings on the C8/C9+ Aromatics Fractionation 16 can result in as much as a 75% reduction in the overall energy consumption of the process for the production of paraxylene.

FIG. 2 shows that the Liquid Phase Isomerate Recycle Stream 21 can be optionally sent to one or more locations which include C8 Aromatics/C9+ Aromatics Fractionation 16, via conduit 50 to Benzene Removal unit 23 (with attendant benzene stream 22) and via conduit 60 directly to Paraxylene Recovery 12. The amount sent to each location is determined by the need to remove by-products which include benzene, and C9+ aromatics. The by-products from Liquid Phase Xylenes Isomerization 20 in the Liquid Phase Isomerate Recycle Stream 21 may need to be removed down to a level that is acceptable for Paraxylene Recovery 12, especially if selective adsorption is used for recovering paraxylene. The C9+ aromatics can be removed in the C8/C9+ Aromatics Fractionation 16 or in one or more devices that employ separation techniques such as membrane, extraction, and adsorption. Similarly, benzene can be removed using one or more devices that employ separation techniques such as distillation, extraction, membrane, and adsorption. Optionally, the C9+ aromatics and benzene can be removed simultaneously using one or more devices that employ separation techniques such as distillation, extraction, membrane, and adsorption.

Optionally, in the presence of hydrogen in stream 9, Vapor Phase Xylenes Isomerization 13 establishes a near-equilibrium balance of xylene isomers in stream 19 using one or more of a variety of catalysts, per se well-known in the art, which may also convert ethylbenzene to benzene and ethane or may convert ethylbenzene to near-equilibrium xylene isomers. The xylenes isomerization product stream 19 passes to detoluenization fractionation 18 which removes C7 and lighter materials in stream 11 to yield isomerate recycle stream 10. Isomerate recycle stream 10 is processed in the OX and C9+ aromatics removal unit 16.

Regarding separation of xylenes in the PX recovery, two preferred methods are fractional crystallization and selective adsorption, the details of which are per se known in the art. See, for instance, in this regard, for example, U.S. Pat. No. 7,439,412, and also references cited in the Background section above. The details of crystallization and selective adsorption are not per se the subject of the present invention.

Likewise, the details of vapor phase xylenes isomerization and liquid phase xylenes isomerization are also per se known in the art. In this regard, see for example, U.S. Pat. Nos. 6,180,550; 6,448,459; 6,872,866; 7,244,409; 7,371,913; 7,495,137; 7,592,499; U.S. Patent Application Publication No. 2009-0182182; U.S. application Ser. No. 12/612,007; and Provisional Patent Application No. 61/326,445, filed Apr. 21, 2010.

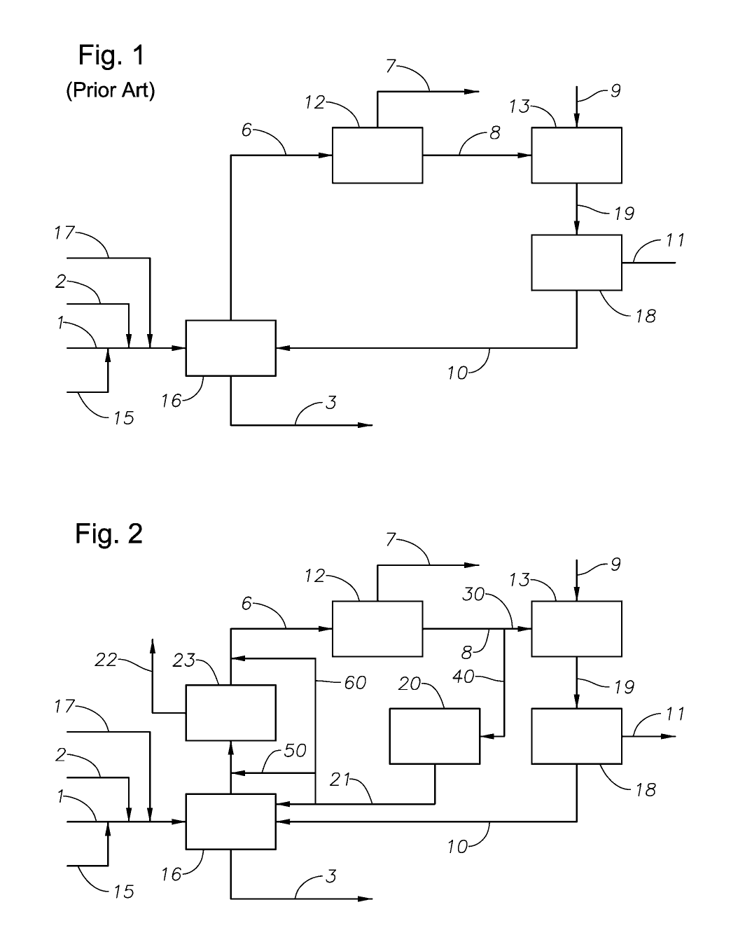

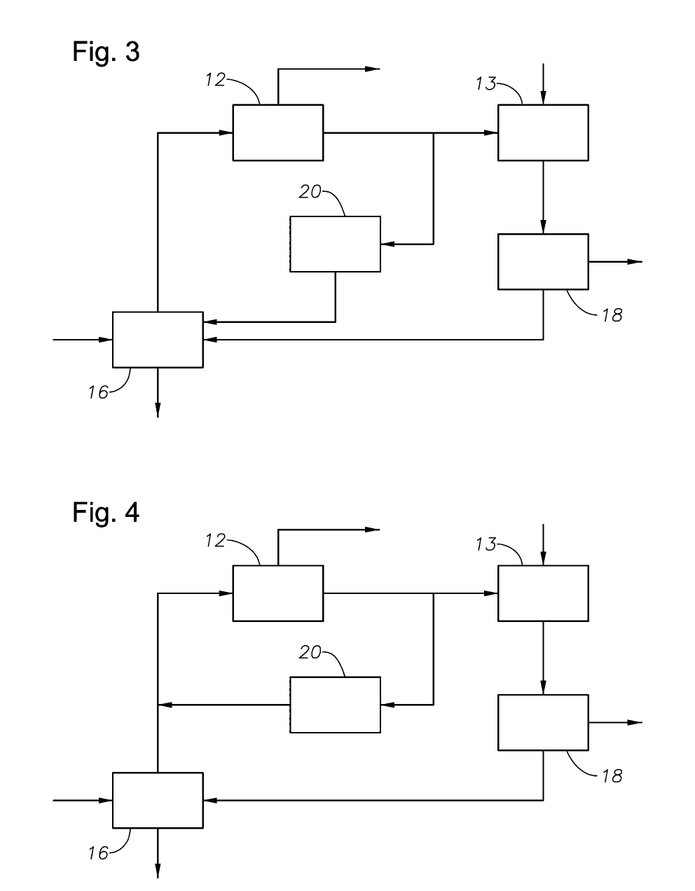

Computer simulations using the Pro II program, which is commercially available is software, were conducted to verify the benefits of the present invention. Appropriate assumptions were made, such as provided in U.S. Pat. No. 7,439,412, which are within the skill of the ordinary artisan. A plant with a PX capacity of 540 kta served as the base case (FIG. 1). The simulations studied two process arrangements: Process A shown in FIG. 3, and Process B shown in FIG. 4. In both Processes A and B, the PX-depleted stream of C8 aromatics from PX recovery unit 12 was split into two equal fractions, one of which was sent to a Liquid Phase Xylenes Isomerization unit 20, while the other fraction is sent to Vapor Phase Xylenes Isomerization unit 13. As shown in the figures, Process A (FIG. 3) sent the product from the Liquid Phase Xylenes Isomerization unit 20 to the rerun tower 16, while Process B (FIG. 4) sent the product from Liquid Phase Xylenes Isomerization unit 20 to the PX recovery unit 12 (a Parex™ absorptive separation unit, per se well-known in the art). As in FIGS. 1 and 2, both FIGS. 3 and 4 have the vapor phase isomerizate from 13 pass through detoluenizer 18 to take out toluene and pass equilibrium or near equilibrium xylenes back to rerun tower 16. The simulations show that using the process according to the invention, there are significant energy savings of 13.10 MW (mega Watts) for Process B (FIG. 4) and 12.45 MW for Process A (FIG. 3) compared to the base case of FIG. 1.

The invention has been described above with reference to numerous embodiments and specific examples. Many variations will suggest themselves to those skilled in this art in light of the above detailed description.

In another embodiment, this invention relates to:

1. A process for producing paraxylene comprising:

(a) separating a feed comprising C8+ aromatics in a first fractionation step into a first stream comprising xylene isomers and a second stream comprising C9+ aromatics;

(b) separating said first stream in a paraxylene (PX) recovery unit to recover a PX-rich stream and a PX-depleted stream;

(c) passing a first portion of said PX depleted stream through a vapor phase xylenes isomerization unit to produce a first isomerate stream and a second portion of said PX-depleted stream through a liquid phase xylenes isomerization unit to produce a second isomerate stream; and

(d) optionally further comprising a benzene separation step, wherein benzene is separated from a stream comprising xylenes.

2. The process of paragraph 1, including at least one benzene separation step selected from a benzene separation step between said liquid phase xylenes isomerization unit and said is first fractionation step, and a benzene separation step between the liquid phase isomerization unit and said PX recovery unit.
3. The process of one of paragraphs 1 and 2, wherein said second isomerate stream is recycled to one or more of said first fractionation step, one or more benzene separation step(s) (where present), and said PX recovery unit.
4. The process of any one of the preceding paragraphs, wherein at least a portion of product of said liquid phase isomerization unit is recycled to said first fractionation step.
5. The process of any one of paragraphs 2 to 4, wherein at least a portion of product of said liquid phase isomerization unit is recycled to at least one benzene separation step(s).
6. The process of any one of the preceding paragraphs, wherein a product of said liquid phase isomerization unit is recycled to said PX recovery step.
7. The process of any one of the preceding paragraphs, wherein said PX recovery step includes a crystallization unit.
8. The process of any one of the preceding paragraphs, wherein said PX recovery step includes selective adsorption.
9. The process of any one of the preceding paragraphs, further including a step of fractionation of an isomerate recycle stream to remove toluene, if present in said stream.
10. The process of any one of the preceding paragraphs, wherein said feed comprising C8+ aromatics includes at least one feed selected from the group consisting of a C8+ selective toluene disproportionation product, a C8+ transalkyation product, a C8+ reformate product, and a C8+ toluene disproportionation product.
11. In an apparatus for the production of paraxylene (PX) comprising a first fractionation column operating at conditions suitable for the separation of a C8+ aromatics stream into an overheads comprising xylenes and a bottoms product stream comprising C9+ aromatics, the overheads stream fluidly connected with a PX recovery unit, wherein said PX recovery unit provides a PX-enriched stream and a PX-depleted stream, the improvement comprising dividing a conduit carrying said PX-depleted stream so that a portion of said PX-depleted stream is passed to a vapor phase isomerization unit and another portion of said PX-depleted stream is passed to a liquid phase isomerization unit.
12. The apparatus of paragraph 11, wherein said liquid phase isomerization unit is fluidly connected so as to provide liquid phase isomerate recycle to said first fractionation column and/or to said PX recovery unit.
13. The apparatus of paragraph 11, wherein said PX recovery unit is selected from at least one of a crystallizer and an adsorptive separator.
14. The apparatus of paragraph 11, further including at least one other fractionator upstream of said first fractionator, wherein said at least one other fractionator operates under conditions suitable for removing benzene from a stream comprising xylenes or for removing toluene from a stream comprising xylenes, or both, and wherein said at least one other fractionator is upstream of said first fractionator.

Trade names used herein are indicated by a ™ symbol or ® symbol, indicating that the names may be protected by certain trademark rights, e.g., they may be registered trademarks in various jurisdictions. All patents and patent applications, test procedures (such as priority documents, ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

What is claimed is:

1. A process for producing paraxylene comprising:
(a) separating a feed comprising C8+ aromatics in a first fractionation step into a first stream comprising xylene isomers and a second stream comprising C9+ aromatics;
(b) separating said first stream in a Paraxylene, PX, recovery unit to recover a PX-rich stream and a PX-depleted stream;
(c) passing a first portion of said PX depleted stream through a vapor phase xylenes isomerization unit to produce a first isomerate stream and a second portion of said PX-depleted stream through a liquid phase xylenes isomerization unit to produce a second isomerate stream; and
(d) optionally further comprising a benzene separation step, wherein benzene is separated from a stream comprising xylenes.

2. The process of claim 1, including at least one benzene separation step selected from a benzene separation step between said liquid phase xylenes isomerization unit and said first fractionation step, and a benzene separation step between the liquid phase isomerization unit and said PX recovery unit.

3. The process of claim 1, wherein said second isomerate stream is recycled to one or more of said first fractionation step, one or more benzene separation step(s) (where present), and said PX recovery unit.

4. The process of claim 2, wherein said second isomerate stream is recycled to one or more of said first fractionation step, one or more benzene separation step(s) (where present), and said PX recovery unit.

5. The process of claim 1, wherein at least a portion of product of said liquid phase isomerization unit is recycled to said first fractionation step.

6. The process of claim 2, wherein at least a portion of product of said liquid phase isomerization unit is recycled to said first fractionation step.

7. The process of claim 2, wherein at least a portion of product of said liquid phase isomerization unit is recycled to at least one benzene separation step(s).

8. The process of claim 3, wherein at least a portion of product of said liquid phase isomerization unit is recycled to at least one benzene separation step(s).

9. The process of claim 1, wherein a product of said liquid phase isomerization unit is recycled to said PX recovery step.

10. The process of claim 2, wherein a product of said liquid phase isomerization unit is recycled to said PX recovery step.

11. The process of claim 1, wherein said PX recovery step includes a crystallization unit.

12. The process of claim 2, wherein said PX recovery step includes a crystallization unit.

13. The process of claim 3, wherein said PX recovery step includes a crystallization unit.

14. The process of claim 1, wherein said PX recovery step includes selective adsorption.

15. The process of claim 2, wherein said PX recovery step includes selective adsorption.

16. The process of claim 3, wherein said PX recovery step includes selective adsorption.

17. The process of claim 1, further including a step of fractionation of an isomerate recycle stream to remove toluene, if present in said stream.

18. The process of claim 2, further including a step of fractionation of an isomerate recycle stream to remove toluene, if present in said stream.

19. The process of claim 9, further including a step of fractionation of an isomerate recycle stream to remove toluene, if present in said stream.

20. The process of claim 1, wherein said feed comprising C8+ aromatics includes at least one feed selected from the group consisting of a C8+ selective toluene disproportionation product, a C8+ transalkyation product, a C8+ reformate product, and a C8+ toluene disproportionation product.

21. The process of claim 2, wherein said feed comprising C8+ aromatics includes at least one feed selected from the group consisting of a C8+ selective toluene disproportionation product, a C8+ transalkyation product, a C8+ reformate product, and a C8+ toluene disproportionation product.

22. In an apparatus for the production of paraxylene, PX, comprising a first fractionation column operating at conditions suitable for the separation of a C8+ aromatics stream into an overheads comprising xylenes and a bottoms product stream comprising C9+ aromatics, the overheads stream fluidly connected with a PX recovery unit, wherein said PX recovery unit provides a PX-enriched stream and a PX-depleted stream, the improvement comprising dividing a conduit carrying said PX-depleted stream so that a portion of said PX-depleted stream is passed to a vapor phase isomerization unit and another portion of said PX-depleted stream is passed to a liquid phase isomerization unit.

23. The apparatus of claim 22, wherein said liquid phase isomerization unit is fluidly connected so as to provide liquid phase isomerate recycle to said first fractionation column and/or to said PX recovery unit.

24. The apparatus of claim 22, wherein said PX recovery unit is selected from at least one of a crystallizer and an adsorptive separator.

25. The apparatus of claim 22, further including at least one other fractionator upstream of said first fractionator, wherein said at least one other fractionator operates under conditions suitable for removing benzene from a stream comprising xylenes or for removing toluene from a stream comprising xylenes, or both, and wherein said at least one other fractionator is upstream of said first fractionator.
